(19) United States
(12) Patent Application Publication
(10) Pub. No.: US 2018/0228499 A1
(43) Pub. Date: Aug. 16, 2018

(54) CERAMIC BONE CUTTER

(71) Applicant: CeramTec GmbH, Plochingen (DE)

(72) Inventors: Heinrich Alan WECKER, Eckental (DE); Andreas REMPP, Ostfildern (DE); Mateusz JUSZCZYK, Velden (DE)

(21) Appl. No.: 15/749,822

(22) PCT Filed: Aug. 3, 2016
(86) PCT No.: PCT/EP2016/068506

§ 371 Date: Feb. 2, 2018

(30) Foreign Application Priority Data
Aug. 4, 2015 (DE) 10 2015 009 857.8

(51) Int. Cl. A61B 17/16

(52) U.S. Cl. CPC A61B 17/1637; A61B 2017/0088

(57) ABSTRACT

The invention relates to a cutting broaching tool (1) for intraoperatively obtaining bone material from a patient, comprising a main part (3) which rotates about a rotational axis (2) for securing on a rotating tool support and comprising at least one blade (4) which is rigidly connected to the main part (3). The aim of the invention is to prevent the tissue from reacting when using the broaching tool and thus prevent an increased risk of infection, to prevent a thermal load on the bone, to receive the bone material in the interior of the broaching tool, and to discharge the bone material to an external device. This is achieved in that the main part (3) has a cylindrical shape, is hollow, and surrounds a first cavity (5). The blade (4) or all the blades (4) are formed integrally with the main part (3), extend in the direction of the rotational axis (2) of the main part (3) at a distance from an end face (6) of the main part (3), and surround a second cavity (7). The first cavity (5) is connected to the second cavity (7) in order to receive and transport the cut bone material, and the broaching tool (1) is made of a sintered ceramic material.

The invention relates to a cutting broaching tool for intraoperatively obtaining bone material from a patient, comprising a main part which rotates about a rotational axis for securing on a rotating tool support and comprising at least one blade which is rigidly connected to the main part.

In certain application cases during surgical operations on the human body, the patient's own bone material, known as bone chips, is required. It is known that a metal broaching tool is used for this that comprises a main part that rotates about its own rotational axis for securing on a rotating tool support and comprising at least one blade which is rigidly connected to the main part.

A disadvantage of the known broaching tools is that tissue reactions with the broaching tool can occur and an increased risk of infections exists as a result of this and a thermal load on the bone can be caused due to the friction during cutting. Furthermore, the broaching tool should be reusable following sterilization.

The object of the invention is to propose a cutting broaching tool in accordance with the generic term of Claim 1, which prevents the indicated disadvantages. Furthermore, the broaching tool should receive the bone material inside and also have the possibility of transporting it to an external device.

According to the object of the invention, this task is achieved by means of a cutting broaching tool in accordance with the characteristics of Claim 1.

Because the main part has a cylindrical shape and is hollow, and surrounds a first cavity, because the blade or all of the blades are formed integrally with the main part, extend in the direction of the rotational axis of the main part at a distance from an end face of the main part, and surround a second cavity, because the first cavity is connected to the second cavity in order to receive and transport the cut bone material, and because the broaching tool is made of a sintered ceramic material, tissue reactions are prevented because sintered ceramic materials are inert. The risk of infection is significantly less compared with metal broaching tools. Ceramic surfaces facilitate sterilization, ensuring that the broaching tool can be reused. The embodiment of the broaching tool with the two interior cavities that merge with each other makes it possible to remove the bone material or the bone chips. This is particularly advantageous in the case of an increased volume of bone material.

Using the sintered ceramic materials dramatically increases the hardness of the broaching tool compared with known metal broaching tools. This results in the blade's longer service life and a clean cut. The limited friction results in a reduced thermal load on the bone. Tissue reactions are ruled out.

The broaching tool is preferably comprised of a sintered oxide or nitride ceramic. This has proven its worth in joint surgery.

To facilitate easier incision, at least one of the blades extends beyond the rotational axis for submerging into the bone and so cuts beyond the rotational axis.

In one embodiment, the blade or all of the blades open into an annular tip with a transverse bore and the transverse bore is connected with the second cavity. The annular tip gives the blades the necessary stability and facilitates a blade cutting beyond the rotational axis.

To ensure an even incision when cutting and no abrupt engaging of blade to blade occurs when cutting, the blades are curved in the rotational direction of the broaching tool and thus only a section of all of the blades cuts simultaneously. All the blades have the same curvature. All of the blades have the same contour.

To secure the main part to a tool support, it is preferred that a cross bore is arranged in the main part.

In an embodiment, the broaching tool comprises of four blades. Four blades make particularly uniform cutting possible. However, 1 to 6 blades are an advantage depending on the individual application.

Reinforced areas are preferably arranged in the rotational direction behind the cutting edges of the blades.

The broaching tool cuts bones of differing densities using one or more blades. The thickness of the bone chips created is set by means of the geometry of the blades.

The manipulated variables in this case are the blade height and blade length. The broaching tool receives the cut bone chips inside and can eject them by means of a hose-like element connected with the tool.

The broaching tool is characterized by high hardness, high rigidity and biocompatibility. It should be produced using sintered ceramic materials, preferably oxide and nitride ceramic.

The broaching tool comprises a cylindrical main part that is hollow and designed as an interface to the tool support. A preferred embodiment of the blade area is a conical inlet (hollow). The tip comprises in an embodiment blades that narrow to a point and a transverse bore.

In the following the invention is described further using a preferred embodiment of a broaching tool that is the object of the invention.

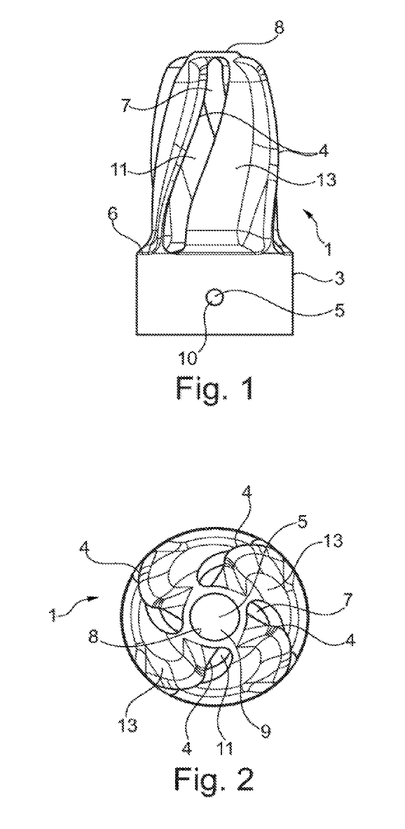

FIG. 1 shows an external view of a broaching tool that is the object of the invention and

FIG. 2 shows a top view, meaning from the point of view of the rotational axis 2.

The broaching tool 1 comprises a cylindrical main part 3 that surrounds a first cavity 5. The main part 3 is open top and bottom. A transverse bore 10 is arranged in the wall of the main part for securing on a tool support not shown here.

Blades 4 are arranged on a side of the main part 3 on its end face 6, that are formed integrally with the main part 3. In the embodiment shown here it is four blades 4 that extend in the direction of the rotational axis 2 of the main part 3 at a distance from one of the end faces 6 of the main part 3, and surround a second cavity 7. The first cavity 5 in the main part 3 is connected to the second cavity 7 within the blades 4. In the embodiment shown here both cavities 5, 7 open into one another to receive and transport the cut bone material.

A curved slot 11 is arranged between the blades 4 whose longitudinal sides are formed by the blades 4. The blades 4 are all designed the same, wherein one of the blades 4 extends beyond the rotational axis 2 for submerging into the bone and so cuts beyond the rotational axis 2.

The broaching tool 1 comprises of a sintered ceramic material and is preferably comprised of a sintered oxide or nitride ceramic.

All of the blades 4 open into an annular tip 8 that comprises a transverse bore 9. This transverse bore 9 is connected with the second cavity 7 or opens into this in this embodiment. The radial distance of the blades 4 to the rotational axis 2 narrows continuously from the end face 6 to the tip 8, so that the blades 4 have an approximate conical embodiment.

The blades 4 are curved in the rotational direction of the broaching tool 1 and thus only a section of bone is cut by all of the blades simultaneously.

In the embodiment shown here reinforced areas 13 are arranged in the rotational direction behind the cutting edges of the blades 4.

1. A cutting broaching tool for intraoperatively obtaining bone material from a patient, comprising a main part which rotates about a rotational axis for securing on a rotating tool support and comprising at least one blade which is rigidly connected to the main part, wherein,
a. the main part has a cylindrical shape and is hollow, and surrounds a first cavity,
b. the blade or all of the blades are formed integrally with the main part, extend in the direction of the rotational axis of the main part at a distance from an end face of the main part, and surround a second cavity,
c. the first cavity is connected to the second cavity in order to receive and transport the cut bone material and
d. the broaching tool is made of a sintered ceramic material.

2. A broaching tool according to claim 1, wherein the broaching tool comprises of a sintered oxide or nitrite ceramic.

3. A broaching tool according to claim 1, wherein at least one of the blades extends beyond the rotational axis for submerging into the bone and so cuts beyond the rotational axis.

4. A broaching tool according to claim 1, wherein the blade or all of the blades open into an annular tip with a transverse bore and the transverse bore is connected with the second cavity.

5. A broaching tool according to claim 1, wherein the blades are curved in the rotational direction of the broaching tool and thus only a section of all of the blades cuts simultaneously.

6. A broaching tool according to claim 1, wherein a transverse bore is arranged in the main part for securing on the tool support.

7. A broaching tool according to claim 1, wherein the broaching tool comprises of four blades.

8. A broaching tool according to claim 1, wherein reinforced areas are arranged in the rotational direction behind the cutting edges of the blades.
